(19) United States
(12) Patent Application Publication
(10) Pub. No.: US 2006/0004437 A1
(43) Pub. Date: Jan. 5, 2006

(54) Structurally variable stents

(76) Inventor: Swaminathan Jayaraman, Fremont, CA (US)

Correspondence Address: PAUL D. BIANCO: FLEIT, KAIN, GIBBONS,;GUTMAN, BONGINI, & BIANCO P.L., 21355 EAST DIXIE HIGHWAY, SUITE 115, MIAMI, FL, 33180, US

(21) Appl. No.: 11/156,992

(22) Filed: Jun. 20, 2005

Related U.S. Application Data
(63) Continuation-in-part of application No. 09/941,327, filed on Aug. 29, 2001, now Pat. No. 6,908,480.

(51) Int. Cl. A61F 2/90

(52) U.S. Cl. 623/1.16; 623/1.42

(57) ABSTRACT

The present invention provides a tubular stent including a longitudinal cylindrical base structure having a first end portion, a second end portion, a mid-portion interposed between the first and second end portions. A plurality of linear strut members connect the mid-portion to the first and second end portions, where the first and second end portions has a first pattern and the mid portion has a second pattern different from the first pattern. The second pattern includes a plurality of articulations. Reservoirs are disposed on at least one of the first end portion, the second end portions, or the mid-portion, where the reservoirs include a pharmaceutical agent therein.

### CROSS-REFERENCE TO RELATED APPLICATION

This application is a continuation-in-part of U.S. application Ser. No. 09/941,327 filed on Aug. 29, 2001, the contents of which are herein incorporated by reference.

### BACKGROUND OF THE INVENTION

This invention relates to stents used to support arterial and venous conduits in the human body. More particularly, it refers to a tubular stent having a non-uniform structure along its longitudinal length to provide good flexibility and radial strength.

There are four major classes of stents employed in the prior art. These four major classes of stents are described as follows:

1. Coil Stents are made from a single wire. The wire is bent in various ways and formed into a stent. Examples of this type of stent are those shown in U.S. Pat. Nos. 4,969,458; 4,681,110 and 5,824,056.

2. Slotted Tube Stents are laser cut using a tube of either stainless steel, nickel/titanium alloy (NITINOL), titanium or any other suitable materials. These designs are preprogrammed into a machine language and a laser is used to cut the programs. These stents have a uniform design and a uniform thickness from the beginning to the end of the stent. In other words, the same segment is repeated from one end of the stent to the other. Examples of this type of stent are described in U.S. Pat. Nos. 4,733,665; 4,739,762; 4,776,337 and 4,793,348.

3. Self Expanding Stents are usually braided or knitted with multiple wire filaments such that they have a lower profile when stretched and they expand from a lower profile to a higher profile when unconstrained in the body. These stents are called self-expanding stents and are described in U.S. Pat. No. 4,655,771.

4. Hybrid Stents are similar to slotted tube stents except that they do not have a closed cell structure but have an open cellular structure with flexible interconnections between each segment of the design. These interconnections provide the flexibility while the segments provide the radial strength and other important properties of the stent. Examples of this stent are described in U.S. Pat. Nos. 5,514,154; 5,562,728; 5,649,952 and 5,725,572.

In use, each of the four classes of stents described above are coated as described in various patents as follows:

1. U.S. Pat. No. 5,759,174 describes a balloon that has a radiopaque segment attached to one of the longitudinal ends of the balloon. When the balloon is inflated, the stent is pressed against the ends of the artery and this indicates the center portion of the dilated stenosis. The external radiopaque marker band is typically made from a dense radiopaque metal such as tantalum, gold, platinum or an alloy of those dense metals.

2. U.S. Pat. No. 5,725,572 describes gold plating on the ends of a stent such that the gold plating marks two bands at the ends of a stent. The patentee mentions that the limitation of gold coating is the stiffening of the stent surface. Hence, the gold plating is done only at the ends where the stiffening does not significantly alter the properties of the stent. Also described is another embodiment where only the exterior of the stent is coated with a radiopaque material.

3. U.S. Pat. No. 5,919,126 describes a patent where the body of the stent is formed from a non-radioactive structural material, a radiopaque material coats the body and a beta emitting radioisotope ion is implanted into the radiopaque material.

4. U.S. Pat. No. 5,824,056 describes an implantable medical device formed from a drawn refractory metal having an improved biocompatible surface. The method by which the device is made includes coating a refractory metal article with platinum by a physical vapor deposition process and subjecting the coated article to drawing in a diamond die. The drawn article can be incorporated into an implanted medical device without removing the deposited material.

5. U.S. Pat. No. 5,824,077 describes a stent which is formed of multiple filaments arranged in two sets of oppositely directed helical windings interwoven with each other in a braided configuration. Each of the filaments is a composite including a central core and a case surrounding the core. The core is formed of a radiopaque material while the outer casing is made of a relatively resilient material, e.g., a cobalt chromium based alloy. Alternative composite filaments described in the patent employ an intermediate barrier layer between the case and the core, a biocompatible cover layer surrounding the case, and a radiopaque case surrounding the central core.

6. U.S. Pat. No. 5,871,437 describes a non-radioactive metallic stent which is coated with a biodegradable or non-biodegradable thin coating of less than about 100 microns in thickness which is selected to avoid provoking any foreign body reaction. This coating contains a radioactive source emitting Beta particles with an activity level of approximately one micro curie and on top of this layer is an anticoagulant substance to inhibit early thrombus formation.

7. U.S. Pat. No. 6,099,561 describes a stent having a biocompatible metal hollow tube constituting a base layer having a multiplicity of openings through an open ended tubular wall thereof, the tube constituting a single member from which the entire stent is fabricated. A thin tightly adherent intermediate layer of noble metal overlies the entire exposed surface area of the tube including edges of the openings as well as exterior and interior surfaces and ends of the wall. A third outermost ceramic like layer composed of an oxide, hydroxide or nitrate of a noble metal is formed atop and in adherent relation to an intermediate layer.

8. U.S. Pat. No. 5,722,984 describes a stent which has an antithrombogenic property and contains an embedded radioisotope that makes the coating material radioactive.

9. Other relevant patents that describe the coating technology or coating properties include U.S. Pat. Nos. 5,818,893; 5,980,974; 5,700,286; 5,858,468; 5,650,202 and 5,696,714.

Although some of the above mentioned stents have good flexibility and others have good radial strength, there is no optimum stent in the prior art that has both good flexibility and radial strength together with the ability to retain a useful coating.

### SUMMARY OF THE INVENTION

The present invention describes a fifth class of stents having multiple designs of structurally variable configuration along the longitudinal length of the stent. The stent has one pattern at both ends of the stent to provide optimal flexibility and a different pattern at the mid-portion of the stent to provide optimal radial strength. Alternatively, the stent has one pattern at each end, a different pattern at its mid-portion and a third pattern in-between the mid-portion and each end. The stent has both closed cell and open cell configuration along its longitudinal length and the closed cells and open cells are interlinked with either straight or wavy configurations in a single stent.

A preferred pattern contains at least three different configurations selected from an open cell design, a closed cell design, a straight interlink or articulation and one wavy interlink or articulation along a variable thickness of connecting stents. Because of the variable thickness of the stents, the amount of drug loaded on the stent is varied along with the release characteristics.

The structurally variable stents of this invention usually have a stainless steel or nickel/titanium alloy (NITINOL) base material with two layers of coating together not exceeding ten microns in depth. One layer is an undercoat in direct contact with the base metal both on the inside and outside surface of the base metal. The topmost layer is in contact with the blood. Both the undercoat and top coat are of the same material such as metallic, biological, synthetic material, or polymeric material.

### BRIEF DESCRIPTION OF THE DRAWINGS

The invention can be best understood by those having ordinary skill in the art by reference to the following detailed description when considered in conjunction with the accompanying drawings in which:

FIG. 1 shows a closed cell design of a stent.

FIG. 2 shows a closed cell design of a stent interconnected by a straight bridge.

FIG. 3 shows an exterior design of a closed cell stent.

FIG. 4 shows a design of an open cell stent with a radiopaque coating on one section of the stent.

FIG. 5 shows a design of a coil stent.

FIG. 6 shows a design of a structurally variable stent having an open cell design on the ends and a closed cell design at the center of the stent.

FIG. 7 shows a design of a structurally variable stent with variable thickness of the open and closed cell design.

FIG. 8 shows a design of a structurally variable stent with open cell at the ends and closed cell at the mid-portion and alternating articulations between both the open and closed cell.

FIG. 9 shows a design of a structurally variable stent with both open and closed cell design and the articulations at the end of the closed cell design is an S-shape rather than a W-shape.

FIG. 10 shows a design of a structurally variable stent with both open and closed cell design and alternating articulations at various sections of the stent.

FIG. 11 shows a design of a structurally variable stent with an open cell design at the ends with multiple S-shapes and a straight articulating member and closed cell design and the mid-portion with a complex plus sign articulation.

FIG. 12 shows a design of a structurally variable stent with a circle at a mid-portion of the open cell design.

FIG. 13 shows a design of a structurally variable stent with different wall thickness along the length of the stent.

FIG. 14 shows a cross sectional view of a portion of the structurally variable stent including two coating layers.

FIG. 15 shows a partial view of a section of stent including a plurality of reservoirs therein.

FIG. 16 shows a section view of the partial section of FIG. 15.

FIGS. 17A-17F show reservoir configurations of the stent of FIG. 15.

FIG. 18 shows a design of a structurally variable stent with both open and closed cell designs including reservoirs at various sections of the stent.

### DETAILED DESCRIPTION OF THE INVENTION

The present invention provides a hollow-tubular self support structure composed of a biocompatible material which can be used as a stent to support arterial and venous conduits in the human body. The stent can include one or more patterns of interconnected lattice works which can be connected by strut members. The patterns can be in the form of a “closed” cell or “open” cell design, wherein “closed cell” and “open cell” are terms of art that a person of ordinary skill in the art would readily understand and appreciate what is covered by the recitation of “closed cell” and “open cell.” Specifically, an open cell stent is defined as a stent that has circumferential sets of strut members with most of the curved sections that are not connected by a longitudinal connecting link to an adjacent circumferential set of struts. A closed cell stent has every curved section of every circumferential set of strut members, except at the distal and proximal end of the stent, attached to a longitudinal connecting link. The definitions of “open cell” and “closed cell” are provided, for example, in U.S. Pat. No. 6,540,774, to Fischell et al, entitled Ultraflexible Open Cell Stent.

Referring now to the drawing figures in which like reference designators refer to like elements, there is shown in FIG. 1, a longitudinal sectional view of a stent 10 of the present invention. The stent 10 includes a series of cells 12 which are longitudinal connected in series, where the cells 12 are interconnected by bridge or strut member 14. The longitudinal serial connections of the cells 12 define the stent as a “closed” cell stent.

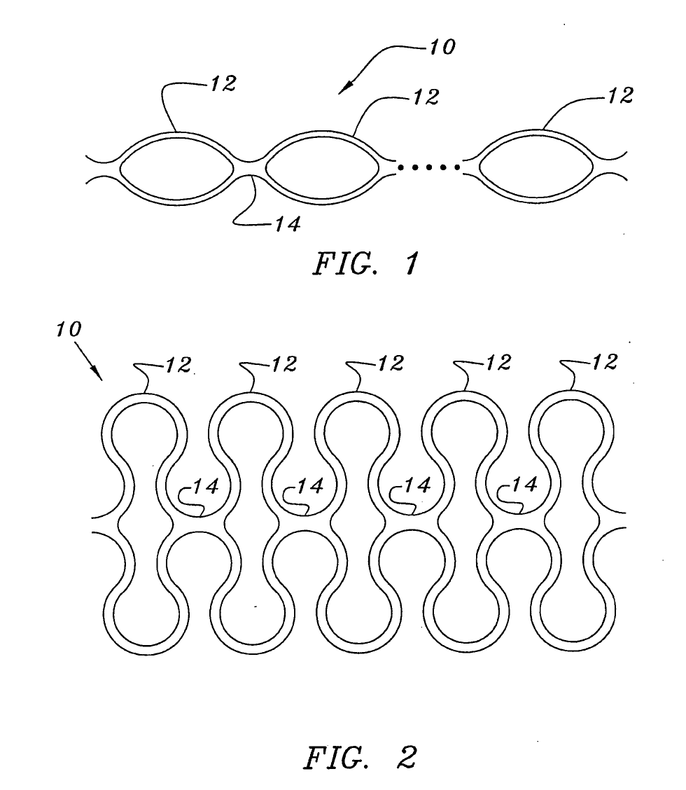

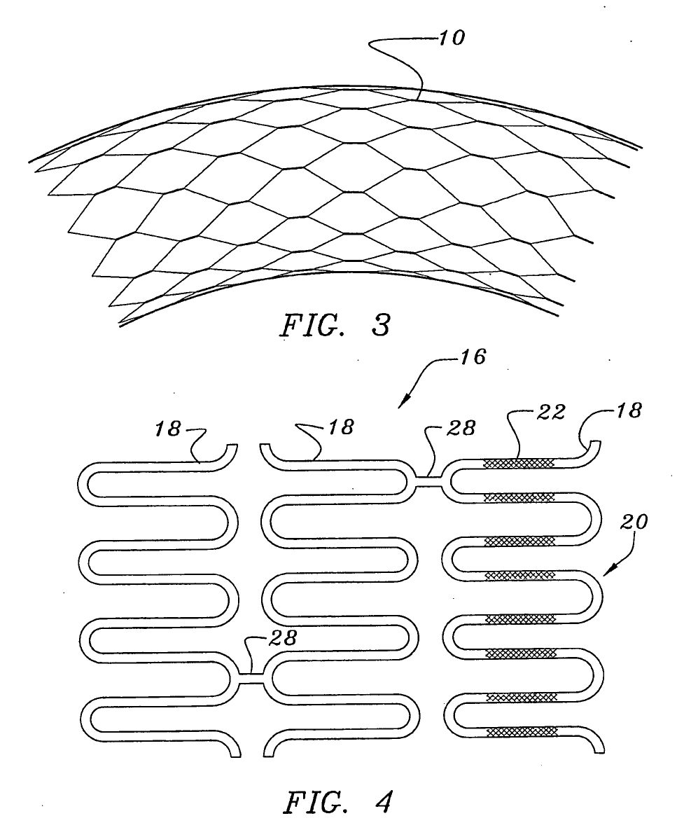

The cells 12 are depicted as having a substantially elliptical shape. However, as shown in FIG. 2, the cells 12 can have a more complex shape. The exterior look of such a stent 10 is provided in FIG. 3.

Referring to FIG. 4, a stent 16 includes a series of cells 18. The cells 18 are shown as circumferential sets of strut members forming an “open” cell stent 16. The circumferential sets of strut members are interconnected with connecting struts 28. Furthermore, at least on one section 20 of the open cell stent 16 can include a radiopaque coating 22 on at a portion of the cell 18. The radiopaque coating 22 can provide an increased visibility of the stent 16 by means of an x-ray, ultrasound, MRI, or other known viewing device.

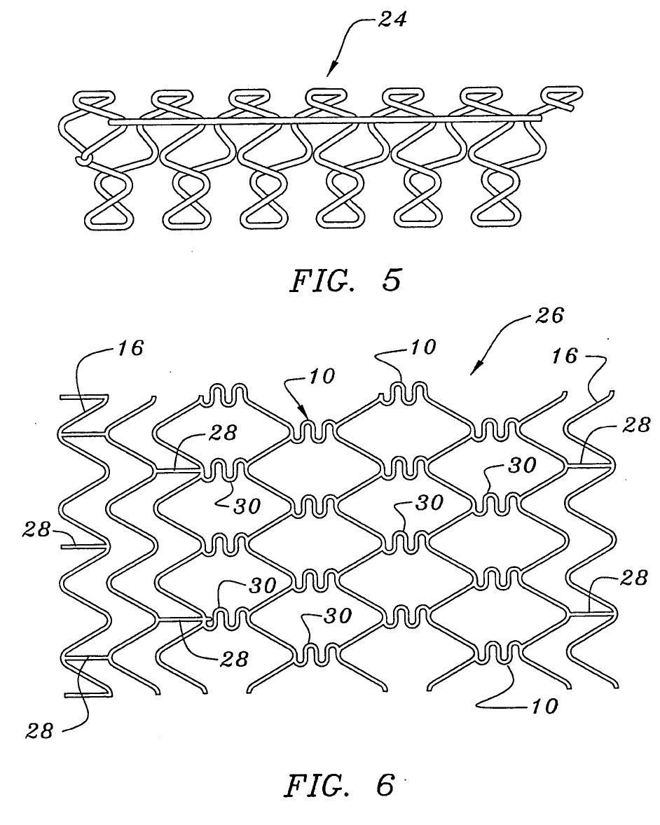

Referring to FIG. 5, a coil stent 24 is provided. A coil stent 24 includes at least one curved segment which is arced about the longitudinal axis of the stent 24.

Referring to FIG. 6, a stent 26 is provided includes a plurality of interconnected cells of differentiating patterns. For example, first and second end portions of the stent 26 have a first pattern 16 and an intermediate portion of the stent 26 has a second pattern 10. The first pattern 16 can be in the form of an open cell configuration and the second pattern 10 be in the form of a closed cell configuration. Connecting struts 28 can join the patterns 10, 16 of the stent 26.

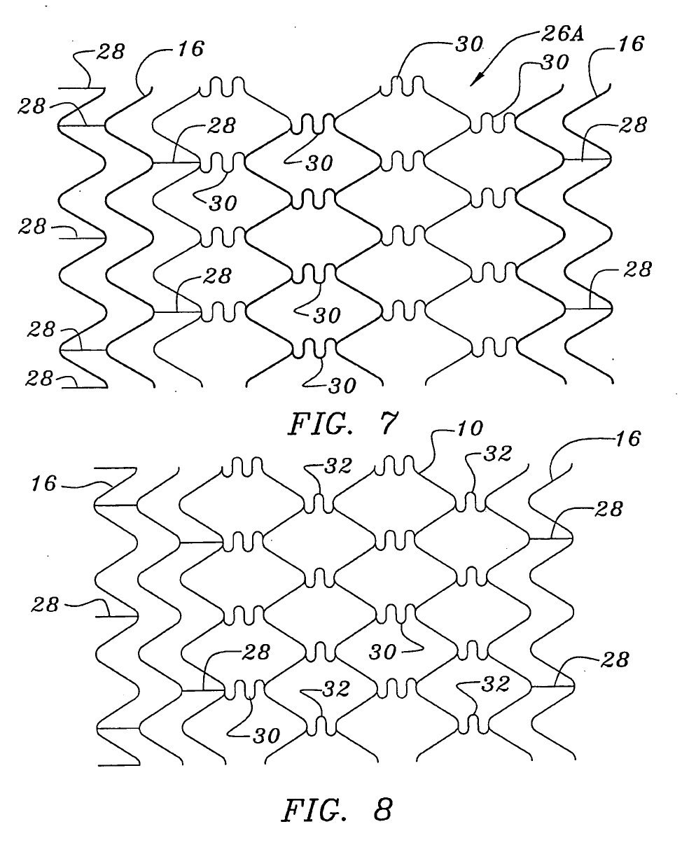

Referring to FIG. 7, a stent 26A is provided. The stent 26A includes a similar structure to stent 26, where the end portions of the stent 26 have an open cell configuration (first pattern) 16 and the intermediate portion of the stent 26 has a closed cell configuration (second pattern) 10. In the stent 26 of FIG. 6, the first and second patterns are depicted as having a uniform material thickness along the length of the stent 26. However, as shown in FIG. 7, the stent 26A can have a varying material thickness along the length of the stent 26A. For example, the first pattern 16 can have a greater material thickness than a material thickness of the second pattern 10. Similarly, the second pattern 10 can have a greater material thickness than the material thickness of the first pattern 16. Alternatively, the material thickness can vary within each of the patterns 10 and 16.

The closed cell configurations 10 further includes articulations 30, where the articulations 30 allow for expansion of the stent 26A. The articulation 30 can be provided in a variety of patterns. For example, the articulations 30 can be provided in a W-pattern. Additional articulation 30 patterns are disclosed in U.S. Pat. No. 6,375,677 to Penn at al, the contents of which are herein incorporated by reference in its entirety.

Referring to FIG. 8, the closed cells 10 can include a plurality of differing shaped articulation. For example, a number of the closed cells 10 can include articulations 30 having a first pattern, the W-pattern, and articulations 32 having a second pattern, an S-pattern.

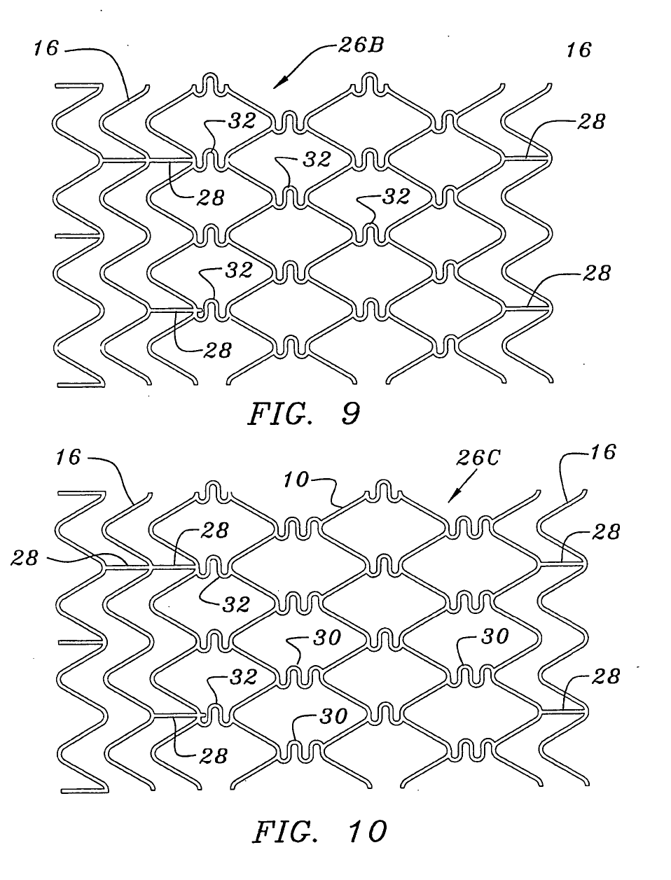

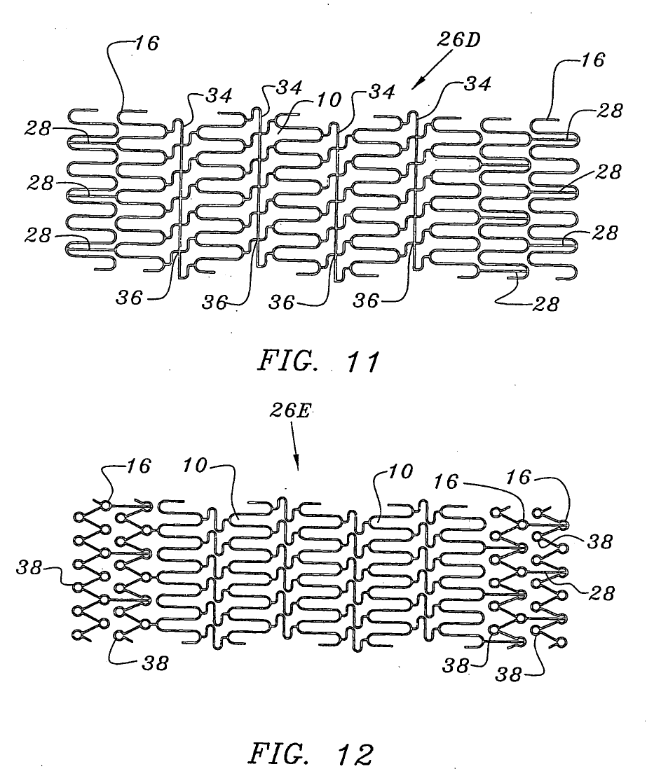

Further, non-limiting, exemplary cell and articulation patterns are provided in the following FIGS. In FIG. 9, the stent 26B has a closed cell design 10 at its mid-portion and an open cell design 16 at each end. The articulations 32 are all in the shape of an S-pattern. In FIG. 10, the stent 26C has a closed cell design 10 at its mid-portion and an open cell design 16 at each end, but with alternating S-pattern 32 and W-pattern 30 articulations. In FIG. 11, the stent 26D has an open cell design 16 at its ends in an S-pattern, a straight articulating member 34, a closed cell 10 mid-portion with a complex plus sign pattern articulation 36. In FIG. 12, the stent 26E has an open cell design 16 at its ends with a circle 38 in the open cell design. The center portion is a closed cell design 10.

Referring to FIG. 13, the stent 26F includes first and second patterns 16 and 10 having varying material thickness. The end portion of the stent 26F includes an open cell configuration. The open cell configuration 16 includes a portion having a thick 40 material thickness and another portion having a thin 42 material thickness. Similarly, the mid portion includes a closed cell configuration 10, which can include portions having a thick 40 material thickness and a thin 42 material thickness. For example, the articulations 32 of the closed cell configuration 10 can have a thick 42 material thickness.

The thickness of the open cell design 16 versus the closed cell design 10 may vary as seen in the drawings. For example, the open cell design 16 can be twenty-five percent thicker than the mid-portion or closed cell design 10.

The combination of an open cell 16 and closed cell 10 stent design creates a stent having both flexibility and radial strength along the length of the stent. The variable stent thickness 40 and 42 provides greater functional properties for coating the stent. If the coating is to enhance the radio opacity, then the ends can be made more radiopaque than the mid-portion. Furthermore, when the stent is coated with a pharmaceutical agent, the thick material can allow for an increased dosage of the pharmaceutical agent to be coated onto the stent. For example, as restenosis occurs in a stent invariably at its ends, a higher pharmaceutical concentration at the ends can more thoroughly inhibit such restenosis.

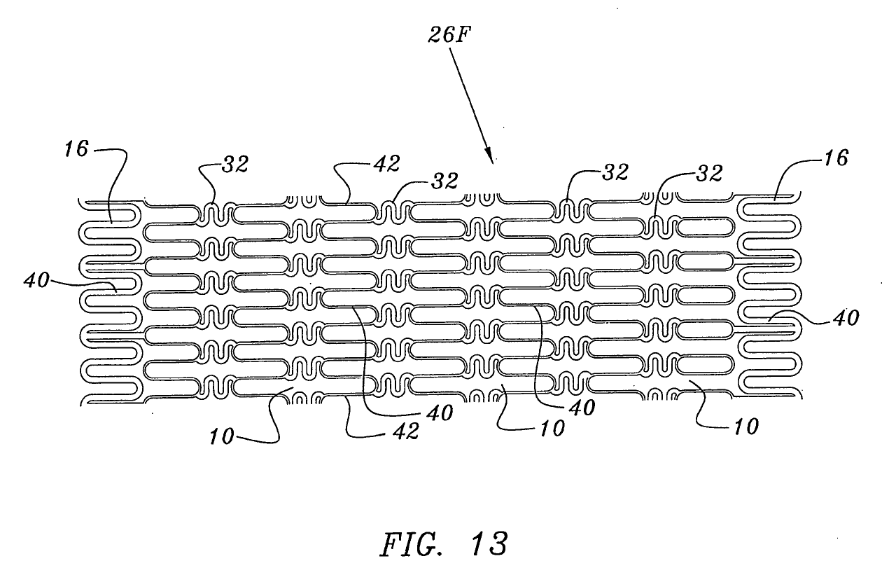

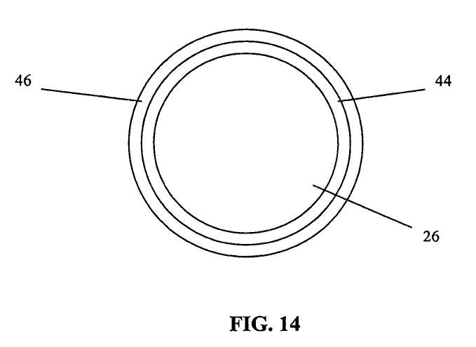

Referring to FIG. 14, the stent 26 can include a plurality of coatings. For example, the stent 26 can include two layers of coatings, a base coat 44 of metal and a top coat 46 of metal enhances radio opacity of the stent 26. Alternatively, the base coat 44 can be a polymeric coating having a top coat 46 which can include a pharmaceutical agent. The pharmaceutical agent can slowly diffuse through the top coat 46 of the stent 26 over a period of time. The variable thickness design of the stents 26-26F can allow for a greater quantity of the pharmaceutical agent to be loaded onto the thick 42 sections of the stent 26, which can facilitate a graded release profile. For example, as noted above, the open cell 16 end portion of the stents 26-26F can have a thick 42 material thickness allowing for a greater quantity of the pharmaceutical agent to be coated onto the end portions of the stents 26-26F.

A coating of at least two layers over the base metal has a depth not to exceed ten microns. Typical coatings are set forth in U.S. Pat. Nos. 5,759,174; 5,725,572; 5,824,056; and 5,871,437 and are herein incorporated by reference.

Referring to FIGS. 15 and 16, the stents 26-26F may include a plurality of reservoirs 48. The reservoirs 48 are dimensioned to receive a pharmaceutical agent 50 therein. The reservoirs 48 are sized to have a volume of at least 1 μg. A coating 52 can be provided to cover the reservoirs 48. The coating 52 can be absorbable or non-absorbable material with the pharmaceutical agent 50 released by diffusing through the coating 52. The coating 52 can be sufficiently permeable to selectively, controllably, release the pharmaceutical agent 50. Alternatively, for an absorbable coating 52, the pharmaceutical agent 50 is released as the coating 52 is absorbed. Alternatively, the coating 52 is coatings 44 and/or 46. The drug 50 is released by slowly diffusing through the coatings 44 and/or 46.

The reservoirs 48 have an opening with a diameter “w” and a depth “d.” The opening of each of the reservoirs 48 may have a uniform diameter “w,” or in the alternative, the opening of each of the reservoirs 48 may have non-uniform diameters “w.”

Similarly, each of the reservoirs 48 may have a uniform depth “d,” or in the alternative, the depth of the each of the reservoirs 48 may be non-uniform. The depth “d” of the reservoir is less than the thickness of the stent material, such that an individual reservoir 48 does not pass completely through the stent material. The reservoir 48 can be formed on the stent by laser cutting, chemical etching, or other related techniques.

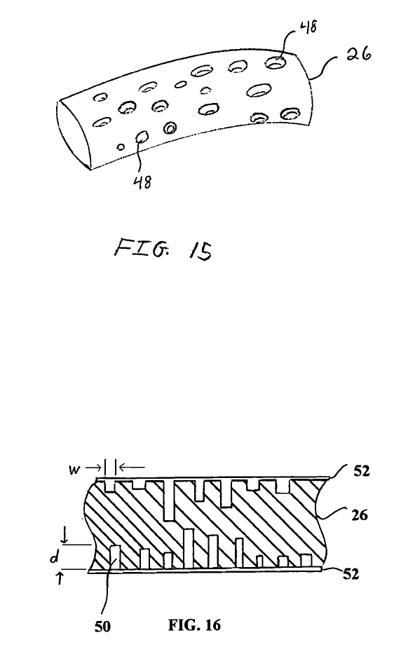

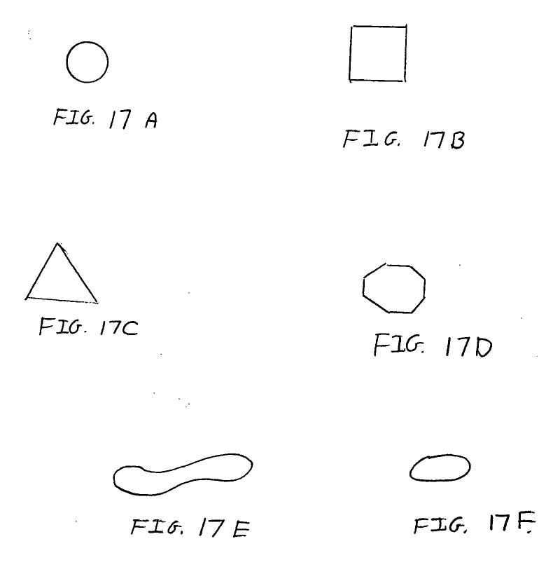

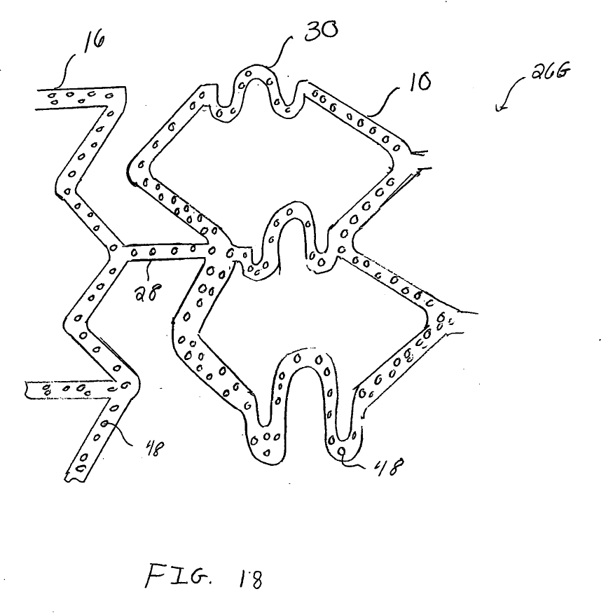

Referring to FIGS. 17A-17F the reservoirs 48 can have circular, elliptical, rectangular, triangular, polygon, or other geometric cross sectional area. Alternatively, the reservoirs 48 can have a free-formed cross-sectional area.

Referring to FIG. 18, the reservoirs 48 can be selectively positioned along the length of the stent 26G. For example, the reservoirs 48 can be positioned in the open cell 16 end portions, the closed cell 10 mid-portion, the articulations 30, the connecting struts, or any combinations thereof. Exemplary configurations include, positioning the reservoirs 48 only on the end portions 16, or only on the mid-portion 10. However, it is contemplated that other reservoir 48 configurations can be utilized.

Additionally, the selectively positioning of the reservoirs 48 further includes controlling the size and density of the reservoirs on each of the stent 26G sections. For example, as restenosis occurs in a stent invariably at its ends, a higher pharmaceutical agent 50 concentration at the ends can more thoroughly inhibit such restenosis. The open cell 16 end portions can have greater reservoir 48 sizes than the closed cell 10 mid-portion of the stent 26G, allowing for a greater pharmaceutical agent 50 concentration to be provide at the end-portions 16 than at the mid-portion 10 of the stent 26G. Alternatively, the open cell 16 end portions can have greater reservoir 48 densities than the closed cell 10 mid-portion of the stent 26G, allowing for a greater pharmaceutical agent 50 concentration to be provided at the end-portions 16 than at the mid-portion 10 of the stent 26G.

It is further contemplated that the reservoirs 48 can be used in combination with the thick 42 and thin 40 materials sections of stent 26-26F. The thick 42 material sections of the stent can allow for increased reservoir 48 sizes and densities to be provided thereon, such that the thick 42 sections of the stent can have a greater pharmaceutical agent 50 concentration than on the thin 40 sections of the stent.

Similarly, the reservoirs 48 can be used in combination with the coating 44 and 46 of FIG. 14. As discussed above, the coatings 44 and 46 can be used to cover the reservoirs 48, wherein the pharmaceutical agent 50 is released by diffusing through the coating 44 and 46. The combination of the coating 44 and 46 and the selective positioning of the reservoirs 48 can be utilized to control the concentration of and release rate of the pharmaceutical agent 50.

As noted above, the coating 46 can similarly include a pharmaceutical agent 50. Where it is desired to have an increased pharmaceutical agent 50 concentration, reservoirs 48 can be provided to be used in combination with the coating 46.

The reservoirs 48 and the coating 46 can include the same pharmaceutical agent 50. Alternatively, the reservoirs 48 and the coating 46 can include different pharmaceutical agents, where the different pharmaceutical agent can be selectively positioned on the stents.

It is additionally contemplated that the reservoirs 48, coatings 44 and 46, and the thick 42 and thin 40 material thickness can be used individually or in combination to control the pharmaceutical agent 50 concentration along the stent.

The stents 26-26G of this invention are longitudinal, cylindrical, metal structures having at least an open cell and closed cell design joined together by struts. The metal can be nickel-titanium alloy (NITINOL) titanium, stainless steel or a noble base metal.

The pharmaceutical agent 50 can include an agent which acts on a calcium independent cellular pathway and may be a macrolide immunosuppressant, or more specifically, rapamycin. Alternatively, the pharmaceutical agent 50 can include an agent to treat or prevent the disease process of the vascular disease. The agent can include an anti-inflammatory agent, non-proliferative agent, anti-coagulant, anti-platelet agent, Tyrosine Kinase inhibitor, anti-infective agent, anti-tumor agent, anti-leukemic agent, or any combination thereof.

Examples of anti-inflammatory agents include, but are not limited to, Zinc compounds, dexarnethasone and its derivatives, aspirin, non-steroidal anti-inflammatory drugs (NSAIDs) (such as ibuprofen and naproxin), TNF-α inhibitors (such as tenidap and rapamycin or derivatives thereof), or TNF-α antagonists (e.g., infliximab, OR1384), prednisone, dexamethasone, Enbrel®, cyclooxygenase inhibitors (i.e., COX-1 and/or COX-2 inhibitors such as Naproxen®, Celebrex®, or Vioxx®), CTLA4-Ig agonists/antagonists, CD40 ligand antagonists, other IMPDH inhibitors, such as mycophenolate (CellCept®), integrin antagonists, alpha-4 beta-7 integrin antagonists, cell adhesion inhibitors, interferon gamma antagonists, ICAM-1, prostaglandin synthesis inhibitors, budesonide, clofazimine, CNI-1493, CD4 antagonists (e.g., priliximab), p38 mitogen-activated protein kinase inhibitors, protein tyrosine kinase (PTK) inhibitors, IKK inhibitors, therapies for the treatment of irritable bowel syndrome (e.g., Zelmac® and Maxi-K® openers), or other NF-κB inhibitors, such as corticosteroids, calphostin, CSAIDs, 4-substituted imidazo[1,2-A]quinoxalines, glucocorticoids, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

Examples of anti-proliferative agents include, but are not limited to, cytochalasins, Taxol®, somatostatin, somatostatin analogs, N-ethylmaleimide, antisense oligonucleotides and the like, cytochalasin B, staurosporin, nucleotide analogs like purines and pyrimidines, Taxol®, topoisomerase inhibitor like topoisomerase I inhibitor or a topoisomerase II inhibitor, alkylating agents such as nitrogen mustards (mechlorethamine, cyclophosphamide, melphalan (L-sarcolysin)), nitrosoureas (carnustine (BCNU), lomustine (CCNU), semustine (methyl-CCNU), streptozocin, chlorozotocin), immunosuppressants (mycophenolic acid, thalidomide, desoxyspergualin, azasporine, leflunomide, mizoribine, azaspirane (SKF 105685)), paclitaxel, altretamine, busulfan, chlorambucil, cyclophosphamide, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, prednisone, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea.

Examples of anti-coagulant agents include, but are not limited to, an RGD peptide-containing compound, heparin, antithrombin compounds, platelet receptor antagonists, anti-thrombin antibodies, anti-platelet receptor antibodies, aspirin, protaglandin inhibitors, platelet inhibitors, tick anti-platelet peptide, hirudin, hirulog, and warfarin.

Examples of anti-platelet agents include, but are not limited to, ReoPro®, ticlopidine, clopidrogel, and fibrinogen receptor antagonists.

Examples of Tyrosine Kinase inhibitors include, but are not limited to, c-Met, a receptor tyrosine kinase, and its ligand, scatter factor (SF), Epithelial Cell Kinase (ECK), inhibitors described in international patent applications WO 96/09294 and WO 98/13350 and U.S. Pat. No. 5,480,883 to Spada, et al., certain 2,3-dihydro-1H-[1,4]oxazino[3,2-g]quinolines, 3,4-dihydro-2H-[1,4]oxazino[2,3-g]quinolines, 2,3-dihydro-1H-[1,4]thiazino[3,2-g]quinolines, and 3,4-dihydro-2H-[1,4]thiazino[2,3-g]quinolines, EGF, PDGF, FGF, src tyrosine kinases, PYK2 (a newly discovered protein tyrosine kinase) and PTK-X (an undefined protein tyrosine kinase).

Examples of anti-infective agents include, but are not limited to Leucovorin, Zinc compounds, cyclosporins (e.g., cyclosporin A), CTLA4-Ig, antibodies such as anti-ICAM-3, anti-IL-2 receptor (Anti-Tac), anti-CD45RB, anti-CD2, anti-CD3 (OKT-3), anti-CD4, anti-CD80, anti-CD86, monoclonal antibody OKT3, agents blocking the interaction between CD40 and CD154 (a.k.a. “gp39”), such as antibodies specific for CD40 and/or CD154, fusion proteins constructed from CD40 and/or CD154/gp39 (e.g., CD40Ig and CD8gp39), β-lactams (e.g., penicillins, cephalosporins and carbopenams), β-lactam and lactamase inhibitors (e.g., augamentin), aminoglycosides (e.g., tobramycin and streptomycin), macrolides (e.g., erythromycin and azithromycin), quinolones (e.g., cipro and tequin), peptides and deptopeptides (e.g. vancomycin, synercid and daptomycin), metabolite-based anti-biotics (e.g., sulfonamides and trimethoprim), polyring systems (e.g., tetracyclins and rifampins), protein synthesis inhibitors (e.g., zyvox, chlorophenicol, clindamycin, etc.), nitro-class antibiotics (e.g., nitrofurans and nitroimidazoles), fungal cell wall inhibitors (e.g., candidas), azoles (e.g., fluoconazole and vericonazole), membrane disruptors (e.g., amphotericin B), nucleoside-based inhibitors, protease-based inhibitors, viral-assembly inhibitors, and other antiviral agents such as abacavir.

Examples of anti-tumor agents include, but are not limited to, DR3 Ligand (TNF-Gamma) and MIBG.

Examples of anti-leukemic agents include, but are not limited to, mda-7, human fibroblast interferon, mezerein, and Narcissus alkaloid (pretazettine).

Examples of chemotherapeutic agents include, but are not limited to, antibiotic derivatives (e.g., doxorubicin, bleomycin, daunorubicin, and dactinomycin), antiestrogens (e.g., tamoxifen), antimetabolites (e.g., fluorouracil, 5-FU, methotrexate, floxuridine, interferon alpha-2b, glutamic acid, plicamycin, mercaptopurine, and 6-thioguanine), cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate), hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone), nitrogen mustard derivatives (e.g., mephalen, chorambucil, mechlorethamine (nitrogen mustard) and thiotepa), steroids and combinations (e.g., bethamethasone sodium phosphate), and others (e.g., dicarbazine, asparaginase, mitotane, vincristine sulfate, vinblastine sulfate, and etoposide).

Examples of anti-angiogenic inhibitors include, but are not limited to, AG-3340 (Agouron, La Jolla, Calif.), BAY-12-9566 (Bayer, West Haven, Conn.), BMS-275291 (Bristol Myers Squibb, Princeton, N.J.), CGS-27032A (Novartis, East Hanover, N.J.), Marimastat (British Biotech, Oxford, UK), Metastat (Aetema, St-Foy, Quebec), EMD-121974 (Merck KcgaA Darmstadt, Germany), Vitaxin (Ixsys, La Jolla, Calif./Medimmune, Gaithersburg, Md.), Angiozyme (Ribozyme, Boulder, Colo.), Anti-VEGF antibody (Genentech, S. San Francisco, Calif.), PTK-787/ZK-225846 (Novartis, Basel, Switzerland), SU-101 (Sugen, S. San Francisco, Calif.), SU-5416 (Sugen/Pharmacia Upjohn, Bridgewater, N.J.), SU-6668 (Sugen), IM-862 (Cytran, Kirkland, Wash.), Interferon-alpha, IL-12 (Roche, Nutley, N.J.), and Pentosan polysulfate (Georgetown University, Washington, D.C.).

Other therapeutic agents include thrombolytic agents such as tissue plasminogen activator, streptokinase, and urokinase plasminogen activators; lipid lowering agents such as antihypercholesterolemics (e.g. HMG CoA reductase inhibitors such as mevastatin, lovastatin, simvastatin, pravastatin, and fluvastatin, HMG CoA synthatase inhibitors, etc.); and anti-diabetic drugs, or other cardiovascular agents (loop diuretics, thiazide type diuretics, nitrates, aldosterone antagonistics (i.e. spironolactone and epoxymexlerenone), angiotensin converting enzyme (e.g. ACE) inhibitors, angiotensin II receptor antagonists, beta-blockers, antiarrythmics, anti-hypertension agents, and calcium channel blockers).

In one example of combinatorial therapy, rapamycin may be combined with GLEEVEC®. GLEEVEC® is a compound which is highly selective for PDGFR alpha, Beta-associated v-Abl tyrosine kinase. These compounds are not only able to inhibit acute vascular lesion formation after denudation injury, but also the development of chronic lesions such as those seen in diffused diseases in the vessel wall. GLEEVEC® can be combined with rapamycin standardization and delivered to the vessel wall via an intravascular implant.

As another example, heparin is known to dissolve clots in the vessel wall. By combining heparin with rapamycin, the stent is much less susceptible to clot formation.

In still another example, curcumin (diferuloylmethane), an anti-inflammatory agent from curcuma longa, affects the proliferation of blood mononuclear cells and vascular smooth muscle cells. Curcumin independently inhibited the proliferation of rabbit vascular smooth muscle cells stimulated by fetal calf serum. Curcumin had a greater inhibitory effect on platelet derived growth factor stimulated proliferation than on serum-stimulated proliferation. Curcumin is very useful in the prevention of pathologic changes of atherosclerosis and restenosis. The possible mechanisms of the antiproliferative and apoptic effects of curcumin on vascular smooth muscle cells were studied in rat aortic smooth muscle cell line. Curcumin inhibits cell proliferation, arrested the cell cycle progression and induced cell apoptosis in vascular smooth muscle cells.

Additional pharmaceutical agents as well as methods to apply these agents are set forth in U.S. Pat. No. 6,585,764 to Wright et al, as well as, commonly owned U.S. patent application Ser. No. 10/696,174 entitled Rationally Designed Therapeutic Intravascular Implant Coating and are herein incorporated by reference.

All references cited herein are expressly incorporated by reference in their entirety.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A tubular stent comprising:
a longitudinal cylindrical base structure including a first end portion, a second end portion, a mid-portion interposed between the first and second end portions, and a plurality of linear strut members connecting the mid-portion to the first and second end portions, the first and second end portions having a first pattern and the mid portion having a second pattern different from the first pattern, the second pattern including a plurality of articulations, wherein at least one of the first end portion, the second end portions, or the mid portion includes a plurality of reservoirs therein.

2. A tubular stent as set forth in claim 1, wherein the plurality of reservoirs includes a first pharmaceutical agent therein.

3. A tubular stent as set forth in claim 2, further comprising a surface coating covering the longitudinal cylindrical base structure.

4. A tubular stent as set forth in claim 3, wherein the surface coating includes at least two layers, at least one of the two layers having a second pharmaceutical agent for inhibiting restenosis, with the second pharmaceutical agent having a higher concentration at the first and second end portions than at the mid-portion.

5. A tubular stent as set forth in claim 4, wherein the first and the second pharmaceutical agents are the same.

6. A tubular stent as set forth in claim 4, wherein the at least two layers comprise a material selected from the group consisting of metallic material, biological material, radiopaque material, synthetic material, polymeric material, and combinations thereof.

7. A tubular stent as set forth in claim 3, wherein the at least two layers have a thickness greater on the first and second end portions than on the mid-portion.

8. A tubular stent as set forth in claim 1, wherein the first pattern is an open cell design and the second pattern is a closed cell design.

9. A tubular stent as set forth in claim 1, wherein a first number of reservoirs of the first and second end portions is greater than a second number of reservoirs on the mid-portion.

10. A tubular stent as set forth in claim 1, wherein a first size of the reservoirs on the first and second end portions is greater than a second size of the reservoirs on the mid-portions.

11. A tubular stent as set forth in claim 1, wherein the longitudinal cylindrical base structure includes a thick and a thin portion.

12. A tubular stent as set forth in claim 11, wherein the first and second end portions include the thick portion.

13. A tubular stent as set forth in claim 12, wherein the first and second end portions have a thickness greater than at the mid portion.

14. A tubular stent as set forth in claim 13, wherein the thickness of the first and second end portions is about twenty-five percent greater than a mid-portion thickness.

15. A tubular stent as set forth in claim 11, wherein the mid-portion includes the thick portion.

16. A tubular stent as set forth in claim 14, wherein the articulations include the thick portion.

17. A tubular stent comprising:
a first end portion having a first pattern;
a second end portion having the first pattern;
a mid-portion interposed between the first and second end portions, and having a second pattern different from the first pattern;
a plurality of linear strut members connecting the mid-portion to the first and second end portions;
a plurality of reservoirs located on at least one of the first end portion, the second end portions, or the mid portion, wherein the first and second end portions have a thickness greater then the mid-portion.

18. A tubular stent as set forth in claim 17, further comprising:
a first pharmaceutical agent disposed within the plurality of reservoirs; and
a surface coating.

19. A tubular stent as set forth in claim 18, wherein the surface coating includes a second pharmaceutical agent.

20. A tubular stent as set forth in claim 19, wherein the first pharmaceutical agent and the second pharmaceutical agent are the same.
